(19) United States
(12) Patent Application Publication
(10) Pub. No.: US 2025/0367117 A1
(43) Pub. Date: Dec. 4, 2025

(54) VITAMIN C-ENCAPSULATED LIPOSOME AND PREPARATION METHOD THEREFOR

(71) Applicant: Hankook Liposome Co., Ltd., Busan (KR)

(72) Inventor: Keun Woo LEE, Busan (KR)

(73) Assignee: Hankook Liposome Co., Ltd., Busan (KR)

(21) Appl. No.: 19/107,261

(22) PCT Filed: Aug. 31, 2022
(86) PCT No.: PCT/KR2022/013061

§ 371 Date: Feb. 27, 2025

(51) Int. Cl. A61K 9/1277; A61K 31/375; A61K 47/12; A61K 47/24

(52) U.S. Cl. CPC A61K 9/1277; A61K 31/375; A61K 47/12; A61K 47/24

(57) ABSTRACT

The present invention provies a method for preparing a vitamin C-encapsulated liposome composition, an oral liposome composition prepared according thereto, and an application method thereof, wherein the preparing method comprises the steps of: (i) mixing 10 to 30 wt % of lecithin, 10 to 30 wt % of vitamin C, and 1 to 10 wt % of citric acid in an aqueous solution; (ii) homogenizing the mixture at 5,000 to 7,000 rpm for 10 to 30 minutes at a temperature of 60 to 70° C.; (iii) stirring the homogenate at 30 to 90 rpm for 60 to 120 minutes at a temperature of 60 to 70° C.; and (iv) cooling the stirred mixture.

### TECHNICAL FIELD

The present invention relates to vitamin C-encapsulated liposome and preparation method therefor.

### BACKGROUND ART

Vitamin C is an essential nutrient that must be obtained externally through food or nutritional supplements. However, after ingestion, the effective concentration of vitamin C in target tissues or cells decreases due to dilution in the body, and the time required to reach an effective dose increases.

To address the issue of vitamin C's low absorption rate in the body, various methods have been proposed, including vitamin C injections, mega-dose supplementation, and transdermal administration. One approach is to encapsulate vitamin C within liposomes, which can enhance its bioavailability and help overcome its inherent instability due to oxidation.

Therefore, encapsulating vitamin C in liposomes can be considered a viable method for the production of vitamin C formulations.

There are various methods for preparing liposomes, including the Bangham method, reverse-phase evaporation, ultrasonication, extrusion, homogenization, ether/ethanol injection, and the dehydration-rehydration method. Among these, the Bangham (film) method is the most common and straightforward approach for manufacturing liposomes. In this method, a thin lipid film is formed by evaporating an organic solvent, followed by the addition of an aqueous solution and mixing to obtain a liposome-containing suspension. This technique is effective for small-scale laboratory production of liposomes. However, it presents challenges for large-scale manufacturing due to difficulties in removing the organic solvent used to dissolve phospholipids and limitations related to the reactor size required for lipid film formation.

Another widely used method is the emulsion method, which allows for size control and ensures a uniform particle distribution. However, this method has the drawback of a low encapsulation efficiency of active substances within liposomes.

To address these limitations, The present inventors developed a more efficient and safer liposome preparing method that overcomes the shortcomings of conventional approaches, such as the Bangham method and the emulsion method. Through this innovation, they aimed to produce a liposomal vitamin C formulation with enhanced bioavailability and stability while enabling large-scale production.

### DISCLOSURE

### Technical Problem

The inventors have attempted to develop a novel liposome preparation method that overcomes the disadvantages of the Bangham (film) method, which uses an organic solvent, as well as the low encapsulation rate of the emulsion method. Through this, the inventors aim to provide a liposomal vitamin C preparation with improved encapsulation efficiency, stability, and bioavailability of vitamin C.

### Technical Solution

To solve the problems, the present invention provides a method for preparing a vitamin C-encapsulated liposome composition, comprising the steps of: (i) mixing 10 to 30 wt % of lecithin, 10 to 30 wt % of vitamin C, and 1 to 10 wt % of citric acid in an aqueous solution; (ii) homogenizing the mixture at 5,000 to 7,000 rpm for 10 to 30 minutes at a temperature of 60 to 70° C.; (iii) stirring the homogenate at 30 to 90 rpm for 60 to 120 minutes at a temperature of 60 to 70° C.; and (iv) cooling the stirred mixture. The present invention also provides a liposome composition obtained by the method and its application.

### Advantageous Effects

The method for preparing a liposome composition encapsulating vitamin C, according to the present invention, not only addresses the issues associated with the use of organic solvents in the conventional Bangham method and the low encapsulation efficiency of the emulsion method but also enhances the stability and bioavailability of vitamin C in the resulting liposome composition. Therefore, it can be effectively utilized as an oral vitamin C formulation.

### DESCRIPTION OF DRAWINGS

FIG. 1 is a schematic diagram showing the preparing method of liposomal vitamin C according to the present invention,

FIG. 2 shows the particle size distribution of liposomal vitamin C measured by dynamic light scattering,

FIG. 3 shows particle size and zeta potential according to the change in concentration of liposomal vitamin C,

FIG. 4 shows particle size (A) and zeta potential (B) according to the change in concentration of liposomal vitamin C and the passage of time, and

FIG. 5 shows the vitamin C concentration in blood samples of experimental animals.

### BEST MODES OF THE INVENTION

Unless otherwise defined, all technical and scientific terms used herein have the same meaning as commonly understood by one of ordinary skill in the art to which this invention belongs. In general, the nomenclature used herein is well known and commonly used in the art.

The present inventors have endeavored to develop a method for producing a liposome preparation with improved physicochemical properties, as well as enhanced stability and bioavailability of vitamin C. This method aims to overcome the shortcomings of conventional liposome production techniques, such as those that use organic solvents (e.g., the Bangham method) or emulsion methods with low liposome encapsulation efficiency. To achieve this, the inventors have developed a novel approach for producing liposomes using only purified water (aqueous solution) and selecting an appropriate surfactant (anhydrous citric acid) in place of conventional methods that rely on organic solvents. More specifically, the liposome preparation method developed by the present inventors is characterized by a process that includes thoroughly dispersing liposomes using a homogenizer (high-speed rotating homomixer), maintaining the temperature of the reactants during stirring (approximately 65° C.), and subsequently cooling the reactants after stirring.

Specifically, the present invention provides a method for preparing a vitamin C-encapsulated liposome composition, comprising the steps of: (i) mixing 10 to 30 wt % of lecithin, 10 to 30 wt % of vitamin C, and 1 to 10 wt % of citric acid in an aqueous solution; (ii) homogenizing the mixture at 5,000 to 7,000 rpm for 10 to 30 minutes at a temperature of 60 to 70° C.; (iii) stirring the homogenate at 30 to 90 rpm for 60 to 120 minutes at a temperature of 60 to 70° C.; and (iv) cooling the stirred mixture.

In the present invention, liposome refers to a single-layer or multi-layer lipid-bilayer structure most similar to a cell membrane having a matrix form of a phospholipid bilayer. Liposomes can be molecular groups that form a bilayer closed membrane composed of phosphatidylcholine (PC), ethanolamine (PE), serine, sphingomyelin, cardiolipin, plasmogen, phosphatidylic acid, cerebroside, etc., and are in equilibrium with water. Phospholipids, which are the basic units constituting liposomes, are composed of an anionic or polar head portion and two nonpolar hydrocarbon chains. The hydrocarbon chains vary in length, and in the case of natural phospholipids, the hydrocarbon chains are 16 or more in length and have an unsaturation degree of about 1 pair. Liposomes are natural or synthetic phospholipids that participate in metabolism, are interchangeable with biological membrane molecules, are biodegradable, have little toxicity to the body, and can capture active substances without chemical bonding.

In the present invention, the liposome encapsulating vitamin C refers to a liposome in which vitamin C is encapsulated inside, and is also called liposomal vitamin C. In general, the bioavailability of vitamin C is greatly affected by the absorption rate in the small intestine after ingestion and the renal reabsorption and excretion rates (see e.g., Padayatty, Sebastian J., et al.), and it is known that liposomes, as effective delivery vehicles for drugs or nutrients, can contribute to improving the bioavailability of the delivery substances (literature [Davis, Janelle L., et al.], etc.). Therefore, liposomal vitamin C not only improves bioavailability compared to oral administration of vitamin C alone, but also has the advantage of improving the stability of vitamin C itself.

The preparation method of the present invention comprises the step of (i) mixing 10 to 30 wt % of lecithin, 10 to 30 wt % of vitamin C, and 1 to 10 wt % of citric acid in an aqueous solution.

The essential ingredients included in the method for preparing liposomal vitamin C of the present invention are lecithin, vitamin C and citric acid, and the weight % included therein is based on the weight of the total composition.

In the present invention, lecithin is one of the phospholipids containing glycerol phosphate. Lecithin is a major component of biological membranes, and is abundantly contained in animal brains, spinal cords, blood cells, egg yolks, etc., and plant seeds, yeast, molds, etc. An example of lecithin is phosphatidyl choline (PC), which is characterized by having hydrophilic components such as phosphoric acid and choline bound to one side of glycerol, and a hydrophobic acyl group bound to the other side.

In the present invention, lecithin may be, without limitation, hydrogenated lecithin, unsaturated lecithin, lyso lecithin, etc. In addition, extracted lecithin (egg yolk lecithin, soybean lecithin, etc.), synthetic lecithin, or a combination thereof may be used.

In the present invention, lecithin is included in an amount of 10 to 30 wt % based on the total weight of the composition. If lecithin is included in an amount of less than 10 wt %, there is a disadvantage in that the absorption rate is lowered because the rate of liposomalization of vitamin C is low, and if it is included in an amount of more than 30 wt %, there are disadvantages such as the formation of aggregates or discoloration. Preferably, lecithin in the present invention may be included in an amount of about 15 to 25 wt % based on the total weight of the composition, more preferably about 20 wt %.

In the present invention, vitamin C refers to ascorbic acid or a salt thereof, and may be extracted vitamin C, synthetic vitamin C, or a combination thereof.

In the present invention, vitamin C is included in an amount of 10 to 30 wt % based on the total weight of the composition. If vitamin C is included in an amount exceeding 30 wt %, there are disadvantages such as vitamin C not dissolving when the reaction product is cooled but precipitating in a solid phase. Preferably, vitamin C in the present invention may be included in an amount of about 15 to 25 wt %, more preferably about 20 wt %, based on the total weight of the composition.

In the present invention, citric acid is a weak organic acid component also called citric acid, and is an essential component for forming liposomes of the liposomal vitamin C of the present invention. Citric acid helps to form liposomes well by the affinity between the hydrophilic head and hydrophobic tail of phospholipids after lecithin is dispersed in an aqueous solution in the preparing method of the present invention. The present inventors have confirmed for the first time that by selecting citric acid among surfactants in the preparing method of liposomes, the physicochemical properties of conventional liposomes, the stability and bioavailability of vitamin C preparations, etc. can be improved.

In the present invention, citric acid may be hydrous citric acid, anhydrous citric acid, or a combination thereof, and among them, anhydrous citric acid is preferable.

In the present invention, citric acid is included in an amount of 1 to 10 wt % based on the total weight of the composition, taking into account the contents of the aforementioned lecithin and vitamin C. If citric acid is included in an amount exceeding 10 wt %, there is a disadvantage in that the interfacial activity between hydrophobic portions of phosphatidylcholine and water in the phospholipid molecule and the like, which is formed during the liposome formation process by lecithin, significantly decreases, thereby lowering the efficiency of liposome formation. Preferably, citric acid in the present invention may be included in an amount of about 2.5 to 7.5 wt % based on the total weight of the composition, more preferably about 5 wt %.

The preparation method according to the present invention is characterized by the absence of organic solvents in the mixing and homogenization process. Unlike conventional liposome preparation methods that utilize organic solvents, the method of the present invention eliminates concerns regarding toxicity or stability issues associated with residual organic solvents. As a result, the liposomes obtained through this method can be safely used without such concerns.

The preparing method of the present invention is characterized by performing, together with or after step (i), a step (ii) of homogenizing the mixture at a temperature of 60 to 70° C. for 10 to 30 minutes at 5,000 to 7,000 rpm.

In the step (i), the mixed lecithin, vitamin C, and citric acid are homogenized to produce a liposome form in which vitamin C is encapsulated. The homogenization step is performed at a temperature of 60 to 70° C., more preferably at a temperature of about 65° C. If the reaction temperature is too low, it is difficult for lecithin or vitamin C to be solubilized, and if the temperature is too high, vitamin C may be destroyed or the formation of lecithin liposomes may be interfered with. The reaction time may be 10 to 30 minutes, preferably about 20 minutes.

The above homogenization is a process of mixing or emulsifying two or more substances by applying mechanical force, and may be performed using an agitator, homogenizer, mixer, or stirring method, and is preferably performed using a homogenizer or mixer (e.g., homo mixer).

The preparing method of the present invention is characterized by performing a step of stirring the homogenate at a temperature of 60 to 70° C. for 60 to 120 minutes at 30 to 90 rpm after the step (ii). The stirring step of the present invention is a unique process of the present invention that is performed separately from the homogenization step, and is a process of additionally stirring the liposome composition homogenized by a homogenizer. The stirring step was devised for the purpose of smoothly performing rolling of liposomes in the homogenate using a paddle.

The preparing method of the present invention includes a step of (iv) cooling the stirred material after the homogenization process of step (ii) and the stirring process of step (iii). The cooling step can be performed by cooling the stirred material at ice water temperature for 20 to 40 minutes, thereby producing the desired liposome.

The preparing method of the present invention may further include a step of (v) obtaining liposomes after the (iv) cooling step.

The present inventors measured the encapsulation efficiency (EE %) of vitamin C in liposomes obtained according to the above preparing method and confirmed that the encapsulation rate of vitamin C in liposomes of the present invention was 30% or more (see Example 2).

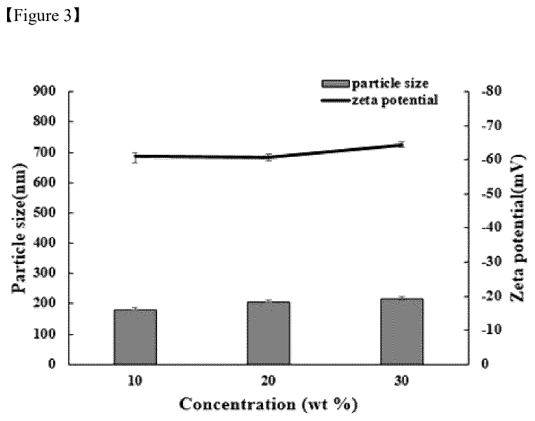

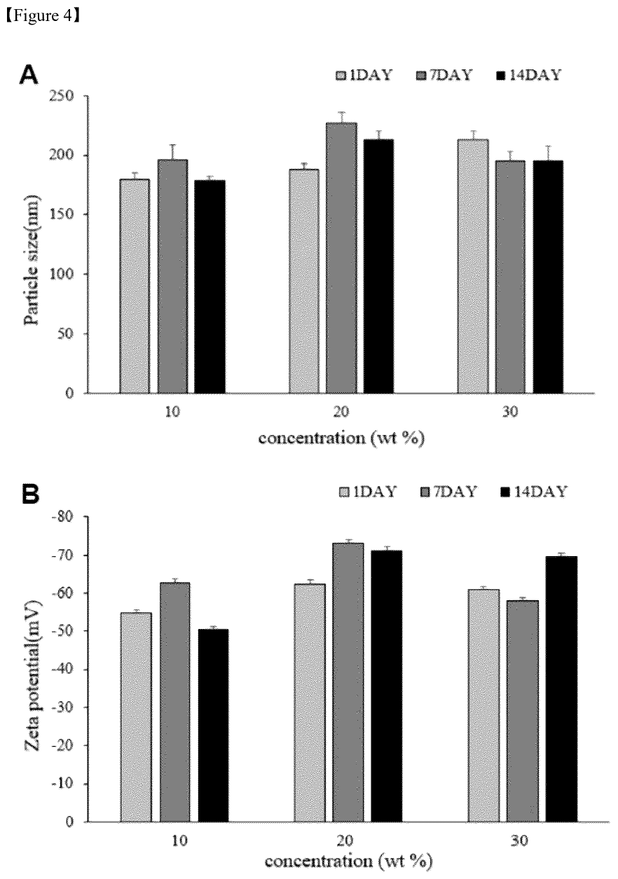

In addition, the present inventors confirmed that the average particle size of the liposomes obtained by the above-described preparing method was 150 to 250 nm and the average zeta potential was as high as −50 to −70 mV (Example 3 and FIGS. 2 and 3). It was confirmed that the physicochemical properties of the liposomes (average particle size and zeta potential, etc.) were maintained even after storage at 25° C. for 14 days after liposome production (Example 4 and FIGS. 4A and B). Therefore, it was confirmed that the liposomes obtained by the preparing method according to the present invention have the advantage of secured stability.

The present inventors also confirmed that the average polydispersity index of liposomes obtained by the above-mentioned preparing method was 0.15 to 0.30, and specifically, about 0.23, so that liposomes of relatively identical size were prepared (Example 3).

The present invention also provides a liposome composition encapsulating vitamin C, according to the above-described preparing method. The liposome composition may be in a liquid state.

The above liposome composition is preferably administered orally and has the advantage of improved bioavailability of vitamin C in the body compared to oral administration of non-liposomal vitamin C.

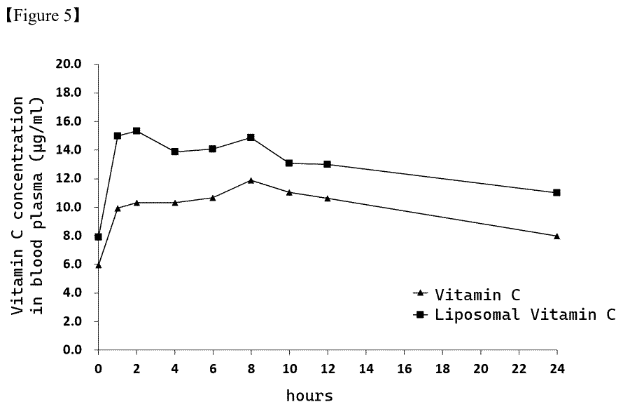

When the liposome composition of the present invention was orally administered to rats, it was confirmed that the blood AUC₀₋₂₄ and Cmax of vitamin C were each improved by more than 30% compared to when non-liposomal vitamin C was administered. Specifically, as a result of analyzing the concentration of blood vitamin C using an experimental animal (SD rat) in the example, the AUC₀₋₂₄ (309.981 μg·hr/ml) and Cmax (16.322 μg/ml) values of the experimental group were measured to be higher than the AUC ₀₋₂₄ (233.957 μg hr/ml) and Cmax (12.017 μg/ml) values of the control group (Example 5, FIG. 5).

As described above, the liposome composition encapsulating vitamin C of the present invention, has an advantage in that, compared to the conventional emulsion method, the liposomes are coagulated and the particle size increases due to the heating effect in the homogenization step using a homogenizer and the stirring step using a paddle, thereby enabling more and more stable encapsulation of vitamin C, thereby improving the bioavailability of vitamin C when actually administered orally, and thus can be usefully utilized as a vitamin C pharmaceutical or health functional food.

The liposome composition encapsulating vitamin C according to the present invention may contain one or more additional additives in addition to water, lecithin, vitamin C, and citric acid. The additives include excipients, stabilizers, preservatives, buffers, etc., which are intended to improve the stability and bioavailability of the composition, maintain the quality of the preparation during preservation or use, and improve economic efficiency by controlling the physical properties of a medicine or health functional food. However, since the technical characteristics of the present invention are not affected by the addition of the additive, it goes without saying that the liposome composition according to the present invention may not contain the additive. In this case, by not containing a specific additive, it may have an additional advantage of not having side effects due to chemical substances that may be caused by the use of the additive.

Liposomal vitamin C (liposomes containing vitamin C) according to the above-described preparing method, can be used as pharmaceuticals or health functional products, depending on their industrial application.

In one aspect, the present invention provides a pharmaceutical composition comprising a liposome encapsulating vitamin C, according to the preparing method described above.

The pharmaceutical composition above can be used to improve diseases or conditions that can be prevented or treated by administering vitamin C to the body. For example, the pharmaceutical composition above can be used to prevent, improve or treat scurvy, wasting diseases in which the demand for vitamin C increases (specifically, decreased physical strength and physical fatigue during pregnancy/lactation and during/after illness) and/or diseases caused by vitamin C deficiency (specifically, capillary bleeding such as gum bleeding, nosebleed, and hematuria, pigmentation caused by sunlight/skin disease, malabsorption, etc.).

The above pharmaceutical composition may be present in various suitable dosage forms for oral administration and may be administered in a pharmaceutically effective amount. Here, the pharmaceutically effective amount means an amount sufficient to treat a disease with a reasonable benefit/risk ratio applicable to medical treatment, and the level of the effective amount may be determined based on factors including the patient's condition, weight, sex, age, health, degree of disease, sensitivity to the drug, time of administration, route of administration, excretion rate, duration of treatment, concurrently used drugs, and other factors well known in the medical field.

The pharmaceutical composition may further comprise one or more active ingredients known to be compatible with vitamin C to maximize the effect of improving the desired disease and/or condition.

In another aspect, the present invention provides a health functional food comprising a liposome encapsulating vitamin C of the above-described preparing method. Here, the health functional food means a food manufactured and processed using raw materials or ingredients having useful functionality for the human body so that, in addition to providing nutrition, it efficiently exhibits a bioregulatory function.

Hereinafter, the present invention will be described in more detail through examples. These examples are only intended to illustrate the present invention, and it will be apparent to those skilled in the art that the scope of the present invention is not to be construed as being limited by these examples.

### EXAMPLE

### Example 1. Preparation of Liposomal Vitamin C

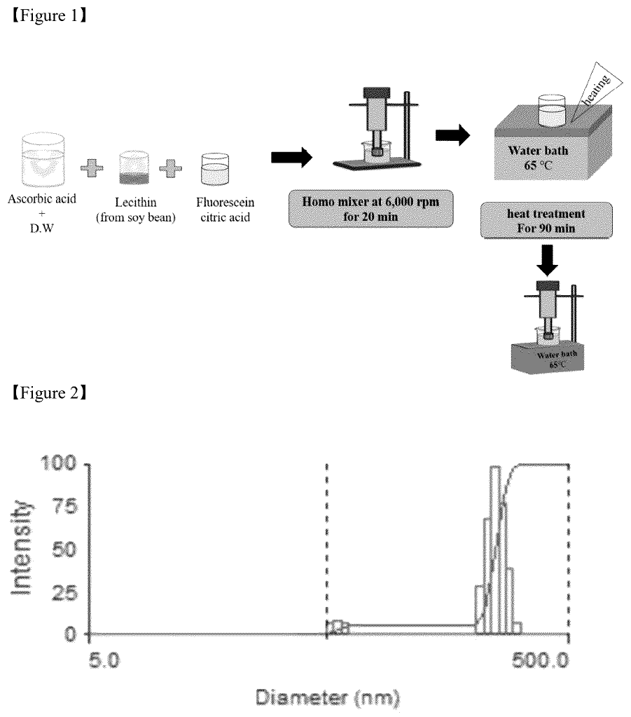

Vitamin C (20 wt %) and anhydrous citric acid (5 wt %) were dissolved in purified water, and lecithin (20 wt %) was added. Then, homogenization and stirring were performed using an in-line disperser (μ-Tron-ISP 25). First, the mixture was homogenized at 65° C. and 6,000 rpm for 20 minutes using a homogenizer (Homo Emulsifier Disperser; HED) to induce dispersion of liposomes. Then, the mixture was stirred at 65° C. and 60 rpm for 90 minutes using a scraper paddle to perform the rolling process of the liposomes. After homogenization was complete, the stirring vessel was immersed in ice water for 30 minutes to prepare liposomes (FIG. 1).

To compare with liposomal vitamin C prepared by a conventional emulsion method, soybean lecithin, vitamin C, and anhydrous citric acid were homogenized at 6,000 rpm for 20 minutes at room temperature to disperse the solution, and liposomes were prepared, which were used as comparative liposomal vitamin C.

### Example 2. Confirmation of Vitamin C Encapsulation Efficiency (EE %) of Liposomes

The various concentrations of liposomal vitamin C solutions prepared in Example 1 were each diluted 10-fold with phosphate buffer saline (PBS), stirred, and the supernatant and precipitate were separated three times at 17,000 rpm for about 20 minutes using a high-speed centrifuge (LZ-1730R, LABOGENE, Seoul, South Korea). Then, each solvent was evaporated using a rotary vacuum concentrator (Rotavapor™ R-300, BUCHI, Switzerland). A small amount of 10% Triton X-100 was added to the container containing the separated and dried liposomes and the container from which the supernatant was evaporated, and stirred. 10% Triton X-100 has the effect of destroying liposomes and releasing the ascorbic acid inside them to the outside. After liposomes were destroyed, they were filtered through a 0.2 μm syringe filter and quantitatively analyzed by high-performance liquid chromatography (HPLC, Agilent 1100, USA). The HPLC analysis conditions for vitamin C were as follows.

TABLE 1

| Column | C18 (4.6 × 150 mm, waters) | |
|---|---|---|
| Column | 28° | C. |
| temperature | | |
| Flow rate | 1 | ml/min |
| Injection | 10 | μl |
| Run time | 15.0 | min |
| Wavelength | 254 | nm |

| | |
|---|---|
| Mobile phase | 0.05M phosphate buffer (pH 7.4)/acetonitrile = 65/35 |

Encapsulation efficiency (EE %) was calculated using the following equation.

$$EE \% = \frac{weight of vitamin C in the liposomes}{weight of feeding vitamine C} \times 100$$

As a result, the vitamin C encapsulation efficiency (EE %) of the liposomes was 38.54 to 38.73%, with an average of 38.67%, which showed a higher encapsulation efficiency than the general vitamin C encapsulation efficiency of liposomes prepared by the homogenization method or the comparative liposomal vitamin C. It is thought that this is because the liposomes aggregate with each other due to the heating effect, increasing the particle size and enabling the encapsulation of more vitamin C.

### Example 3. Measurement of Particle Size, Zeta Potential, and Polydispersity Index of Liposomal Vitamin C

The particle size, zeta potential, and polydispersity index of the liposomal vitamin C at various concentrations prepared in Example 1 were analyzed using a nanoparticle size analyzer (NanoBrook 90Plus PALS, Brookhaven Instruments Corp., NY) based on the principle of dynamic light scattering. Each concentration of liposomal vitamin C solution was diluted 200-fold with distilled water, and measurements were repeated five times in total under the following conditions: refractive index: 1.331, temperature: 25° C., parallel time: 30 sec, measurement angle: 90°, wavelength: 640 nm, and viscosity: 0.890 cP.

As a result, the liposomes had particle sizes of 179 nm to 214 nm (FIG. 2), with an average size of 199 nm, and the polydispersity index (PI) was measured to be 0.189 to 0.275, with an average of 0.227, indicating that the liposomes were distributed in relatively equal sizes. In addition, the zeta potential was measured to be-60.78 to-64.45 mV, with an average of −62.14 mV, indicating a high zeta potential value (FIG. 3). The zeta potential is a numerical value that indicates the degree of stable dispersion between the charges of the particles, and the more the absolute value increases by about −30 mV or more, the more stably the particles are maintained (see Marsanasco, Marina, et al. Food research international 44.9 (2011): 3039-3046.). Therefore, it is confirmed that the liposomal vitamin C of the present invention has a liposome in a stable form.

### Example 4. Stability of Liposomal Vitamin C

As in Example 1, the particle size and zeta potential were measured while storing the liposomal vitamin C at 25° C. for 14 days. As a result, the size of the liposomes did not change significantly over time. Specifically, the particle size was 161 to 237 nm, with an average of 201 nm, and the zeta potential was also −53.53 to −66.52 mV, with an average of −61.25 mV, confirming that the liposomes were stable (FIGS. 4A and B).

### Example 5. Bioavailability of Liposomal Vitamin C

The bioavailability of liposomal vitamin C and conventional vitamin C was compared when administered orally using experimental animals.

The experimental animals used were SD rats (Samtago BIO KOREA Co., Ltd.), which have anatomical and physiological characteristics similar to humans and have the advantage of being useful as a pharmacokinetic model. The SD rats were male, 7-week-old (240-260 g), and were raised at a temperature of 20-24° C. and a relative humidity of 40-70%, and general symptoms were observed once a day for 5 days before the test.

Solid feed for experimental animals (Purina Feed®) was provided, and the rats were allowed to consume it freely. The group composition and administered amount, etc. are as shown in the table below.

TABLE 2

| | | | | No. of |
|---|---|---|---|---|
| Group | Test substance | Dose(ml/kg) | Route | animals |
| G1(control) | Vitamin C | 10 ml(200 mg)/kg | Oral | 6 |
| G2(test) | Liposomal | 10 ml(200 mg)/kg | Oral | 6 |
| | vitamin C | | | |

As a control group, food vitamin C (purity 99.9%) was used, and the weight was measured and dissolved in purified water to prepare it. As a test group, liposomal vitamin C prepared in Example 1 was used, and the encapsulation efficiency (EE %) was calculated to prepare it so that the concentration of the control group and the concentration of vitamin C were the same.

After putting the test substance into a 3 ml syringe, the zonde for rat oral administration was combined, and the test substance was administrated into the rat.

For blood collection, about 300 μl of blood was collected from the jugular vein of all subjects in each group using a disposable syringe (1 ml, 26G, BD Biosciences, USA) at the scheduled time after administration. The blood was placed in a clean, light-shielded 1.5 ml amber tube, allowed to coagulate for 30 minutes at room temperature, and then centrifuged to separate the serum. The oral administration time points were 0, 1, 2, 4, 6, 8, 10, 12, and 24 hours (total of 9 time points).

For blood sample separation and analysis, the harvested whole blood was coagulated and centrifuged at 3,000 rpm for 10 minutes using a centrifuge (Micro17R, Hanil, KOR) to separate the serum. Each 100 μl×1 tube was placed in a 1.5 ml Amber tube labeled with the test type, animal number, and blood collection time and stored in an ultra-low temperature freezer (approximately −70° C.). When testing, the stored samples were thawed at room temperature and the blood concentration of vitamin C was analyzed using HPLC (Alliance e2695, Waters, USA).

TABLE 3

| Column | SunFire C18 (4.6 × 150 mm, 5 μm, Waters) | |
|---|---|---|
| Column temperature | 40° | C. |
| Flow rate | 0.8 | ml/min |
| Injection | 10 | μl |
| Run time | 10.0 | min |
| Wavelength | 245 | nm |
| Autosampler | 10° | C. |
| temperature | | |

| | |
|---|---|
| Mobile phase | 0.1% Formic acid |

For sample processing and analysis, 70 μL of the sample was taken and placed in a microtube, 70 μL of metaphosphoric acid (0.1 g/mL in water) was added, and then mixed using a vortexer for 30 seconds. The mixture was centrifuged at 14,000 rpm for 10 minutes, and the supernatant was transferred to a vial and injected into the HPLC for analysis. Since vitamin C is an endogenous substance, it was used after serum dialysis.

As a result, there were no dead animals during the test period, and there were no unusual events after administration of the test substance. As a result of analysis of biological samples, the pharmacokinetic analysis of vitamin C in the control vitamin C administration group was calculated as AUC₀₋₂₄ of 233.957 μg·hr/ml, Cₘₐₓ of 12.017 μg/ml, Tₘₐₓ of 7.667 hr, and T_{1/2} of 27.759 hr. As a result of pharmacokinetic analysis of vitamin C in the liposomal vitamin C administration group according to the present invention, AUC₀₋₂₄ of 309.981 μg·hr/ml, Cₘₐₓ of 16.322 μg/ml, Tₘₐₓ of 5.500 hr, and T_{1/2} of 76.028 hr were calculated (FIG. 5).

It was confirmed that the vitamin C bioavailability of the liposomal vitamin C according to the present invention is superior to that of vitamin C not encapsulated in liposomes, and is also superior to that of the comparative liposomal vitamin C prepared by a conventional emulsion method.

1. A method for preparing a vitamin C-encapsulated liposome composition, comprising the steps of:
(i) mixing 15 to 25 wt % of lecithin, 15 to 25 wt % of vitamin C, and 5 wt % of anhydrous citric acid in an aqueous solution wherein surfactants other than the anhydrous citric acid are not used in the mixing step;
(ii) homogenizing the mixture at 6,000 rpm for 20 minutes at a temperature of 60 to 70° C.;
(iii) stirring the homogenate at 30 to 90 rpm for 60 to 120 minutes at a temperature of 60 to 70° C. by rolling liposomes in the homogenate using a paddle; and
(iv) cooling the stirred mixture,
wherein wt % refers to the weight percentage based on the total weight of the composition; and
wherein the mixing and homogenizing steps are performed in the absence of organic solvents.

2-4. (canceled)

5. The method of claim 1, wherein the average encapsulation efficiency of vitamin C in the liposome is at least 30%.

6. The method of claim 1, wherein the average particle size of the liposome is 150 to 250 nm, and the average zeta potential of the liposome is −50 to −70 mV.

7. The method of claim 6, wherein the average particle size and average zeta potential are maintained for 14 at least days after preparation.

8. The method of claim 1, wherein the average polydispersity index of the liposome is 0.15 to 0.30.

9-13. (canceled)
